SEQUENCE LISTING (29 sequences)

SEQ ID NO: 1 - DNA, 25 bp, Artificial Sequence
  primer
cataaggcaagagttttggctgaag

SEQ ID NO: 2 - DNA, 25 bp, Artificial Sequence
  primer
ccataaggcaagagttttggctgaa

SEQ ID NO: 3 - DNA, 25 bp, Artificial Sequence
  primer
ataaggcaagagttttggctgaagc

SEQ ID NO: 4 - DNA, 25 bp, Artificial Sequence
  primer
cataargcaagagttttggckgaag

SEQ ID NO: 5 - DNA, 27 bp, Artificial Sequence
  primer
gcagagaggcaattttaggaaccaaag

SEQ ID NO: 6 - DNA, 25 bp, Artificial Sequence
  primer
gcaatgagccaagtaacaaattcag

SEQ ID NO: 7 - DNA, 25 bp, Artificial Sequence
  primer
cctgtctacttgccacacaatcatc

SEQ ID NO: 8 - DNA, 25 bp, Artificial Sequence
  primer
tgttggaaatgtggaaaggaaggac

SEQ ID NO: 9 - DNA, 25 bp, Artificial Sequence
  primer
acaccaaatgaaagattgtactgag

SEQ ID NO: 10 - DNA, 25 bp, Artificial Sequence
  primer
tgaaagattgtactgagagacaggc

SEQ ID NO: 11 - DNA, 20 bp, Artificial Sequence
  primer
agaaatttgtacagaratgg

SEQ ID NO: 12 - DNA, 19 bp, Artificial Sequence
  primer
caatactccagtatttgcc

SEQ ID NO: 13 - DNA, 20 bp, Artificial Sequence
  primer
tcaagacttctgggaagttc

SEQ ID NO: 14 - DNA, 21 bp, Artificial Sequence
  primer
gggaagttcaattaggaatac

SEQ ID NO: 15 - DNA, 19 bp, Artificial Sequence
  primer
ataaatggacagtacagcc

SEQ ID NO: 16 - DNA, 18 bp, Artificial Sequence
  primer
agctggactgtcaatgac

SEQ ID NO: 17 - DNA, 25 bp, Artificial Sequence
  primer
caggatatgttactrayagaggaag

SEQ ID NO: 18 - DNA, 19 bp, Artificial Sequence
  primer
tgcatggacaagtagactg

SEQ ID NO: 19 - DNA, 20 bp, Artificial Sequence
  primer
gccagtggatatatagaagc

SEQ ID NO: 20 - DNA, 22 bp, Artificial Sequence
  primer
ctttagtttgtatgtctgttgc

SEQ ID NO: 21 - DNA, 19 bp, Artificial Sequence
  primer
ctgctggaataacttctgc

SEQ ID NO: 22 - DNA, 23 bp, Artificial Sequence
  primer
ccttctaaatgtgtacaatctag

SEQ ID NO: 23 - DNA, 18 bp, Artificial Sequence
  primer
racaaactcccactcagg

SEQ ID NO: 24 - DNA, 20 bp, Artificial Sequence
  primer
aatggaggttctttctgatg

SEQ ID NO: 25 - DNA, 18 bp, Artificial Sequence
  primer
tttgtctggtgtggtaar

SEQ ID NO: 26 - DNA, 22 bp, Artificial Sequence
  primer
gtggtattcctaattgaacttc

SEQ ID NO: 27 - DNA, 18 bp, Artificial Sequence
  primer
cctaattgaacttcccag

SEQ ID NO: 28 - DNA, 20 bp, Artificial Sequence
  primer
tatgcatcacccacatccag

SEQ ID NO: 29 - DNA, 20 bp, Artificial Sequence
  primer
ggatgtggtattcctaattg

(19) United States
(12) Patent Application Publication
(10) Pub. No.: US 2024/0209463 A1
(43) Pub. Date: Jun. 27, 2024

(54) PRIMER SET FOR ANALYZING HIV-1 DRUG RESISTANCE, KIT COMPRISING SAME, AND ANAYSIS METHOD USING SAME

(71) Applicant: SCL HEALTHCARE, CO., LTD., Gyeonggi-do (KR)

(72) Inventors: Kyoung Ryul LEE, Gyeonggi-do (KR); Young Jin KIM, Gyeonggi-do (KR); Yun Tae KIM, Gyeonggi-do (KR); Ju Young MOON, Gyeonggi-do (KR); Ki Han KO, Gyeonggi-do (KR)

(73) Assignee: SCL HEALTHCARE, CO., LTD., Gyeonggi-do (KR)

(21) Appl. No.: 17/799,654

(22) PCT Filed: Dec. 16, 2020
(86) PCT No.: PCT/KR2020/018471

§ 371 Date: Aug. 12, 2022

(30) Foreign Application Priority Data
Feb. 13, 2020 (KR) 10-2020-0017771

(51) Int. Cl. C12Q 1/70; C12N 15/10; G16B 30/10

(52) U.S. Cl. CPC C12Q 1/703; C12N 15/1096; G16B 30/10; C12Q 2600/106

(57) ABSTRACT

Embodiments of the invention provide a RT-PCR primer set for analyzing HIV-1 drug resistance, the RT-PCR primer set comprising a forward primer selected from the group consisting of SEQ ID NOs: 1 to 6, and a reverse primer presented as SEQ ID NO: 7; a kit comprising same; and a method for analyzing HIV-1 drug resistance by using same.

### CROSS REFERENCE TO RELATED APPLICATIONS

This application claims priority to and the benefit of PCT Patent Application No. PCT/KR2020/018471, titled, “PRIMER SET FOR ANALYZING HIV-1 DRUG RESISTANCE, KIT COMPRISING SAME, AND ANALYSIS METHOD USING SAME,” filed on Dec. 16, 2020, which claims priority to Korean Patent Application Serial No. KR 10-2020-0017771, filed on Feb. 13, 2020, the disclosures of which are incorporated herein by reference in their entirety.

### REFERENCE TO AN ELECTRONIC SEQUENCE LISTING

The contents of the electronic sequence listing (sequencelisting.txt; Size: 4,425 bytes; and Date of Creation: Jan. 11, 2023) is herein incorporated by reference in its entirety.

### BACKGROUND

### Technical Field

Embodiments of the invention relate to a primer set for determining whether an analysis target subject has resistance to an HIV-1 drug, a kit including the same, and an analysis method using the same. More particularly, the present disclosure relates to a primer set for determining the presence or absence of resistance to an HIV-1 drug by analyzing the nucleotide sequence of a specific region of the POL gene of HIV-1 RNA, a kit including the same, and an analysis method using the same.

### Background Art

Human Immunodeficiency Virus (HIV) is a retrovirus that destroys the human immune system. That is, HIV, when onset, progresses to AIDS. All people infected with HIV are called the HIV-infected, and it is reported that the number of new HIV-infected people is around 2,000,000 per year worldwide.

It is known that about 1,000 new cases of HIV infection are reported in Korea each year. Specifically, in 2017, 1,191 people were reported as newly infected with HIV in Korea, of which 1,009 were Koreans and 182 were foreigners. By gender, 1,089 were men and 102 were women, indicating a sex ratio of 10.7:1.

After treatment for HIV-1 infection has been developed, HIV-1 mutations resistant to anti-HIV-1 treatment have emerged, and there is a problem that the existing anti-HIV-1 treatment is no longer effective for HIV-1 infected people who have such an HIV-1 mutation. Accordingly, to prescribe a suitable treatment (i.e., a suitable therapeutic agent) for each HIV-1 infected person, a so-called HIV-1 drug resistance test of detecting the position of the mutation in the HIV-1 gene possessed by the infected person is required.

Currently commercialized HIV-1 drug resistance diagnostic kit products include “ViroSeq HIV-1 Genotyping System” manufactured by Abbott and “HIV genotyping kit” manufactured by Trugene, but these kits are expensive and are not enough in supply. Accordingly, there is a need to develop a new kit being cheaper and having the same or higher performance than the conventional kits.

On the other hand, diagnosis of HIV-1 drug resistance can be performed through nucleotide sequence analysis of the POL gene of HIV-1 RNA, and whether the nucleotide sequence is mutated is determined by observing a portion of the Protease (PR) region, a portion of the Reverse Transcriptase (RT) region, or a portion of the Integrase (IN) region that are included in the POL gene.

As research on the HIV-1 treatment continues, it has been found that mutations exhibiting resistance to anti-HIV-1 drugs also can appear in regions other than the known target regions in the POL gene. Therefore, there is a need for a new kit capable of diagnosing even areas that are not targeted by the commercially available kits.

On the other hand, it is preferable from the viewpoint of time, effort, and cost to analyze each of the PR region, the RT region, and the IN region at the same time through a one-step analysis compared to the case where the PR region, the RT region, and the IN region are analyzed one after another. However, such a one-step analysis method is not currently known. Even with the use of the Abbott's kit, a one-step analysis can be performed only on the PR and RT regions. Accordingly, there is still a need for the development of a new primer set that targets all of the PR, RT, and IN regions in one step for analysis.

### DOCUMENTS OF RELATED ART

### Patent Document

(Patent Document 1) Korean Patent No. KR 10-1605524

(Patent Document 2) U.S. Pat. No. 6,852,491

(Patent Document 3) U.S. Pat. No. 6,232,455

### SUMMARY

### Technical Problem

Accordingly, a first aspect of the present disclosure is to provide a novel RT-PCR primer set for analyzing a sample to determine resistance against a HIV-1 drug, to solve the above problems and satisfy the above needs.

In addition, a second aspect of the present disclosure is to provide an HIV-1 drug resistance analysis kit including the primer set of the first aspect.

In addition, a third aspect of the present disclosure is to provide a method for analyzing a sample to determine resistance against a HIV-1 drug, the sample being collected from an HIV-1 infected person, by using the primer set of the first aspect and/or the kit of the second aspect.

### Technical Solution

A RT-PCR primer set for determination of resistance of a sample against an HIV-1 drug, to achieve the first aspect of the present disclosure, comprises a forward primer selected from the group consisting of SEQ ID NOs: 1 to 6, and a reverse primer set represented by SEQ ID NO: 7.

According to one embodiment of the present disclosure, the primer set simultaneously targets at least a portion of the Protease (PR) region, at least a portion of the Reverse Transcriptase (RT) region, and at least a portion of the Integrase (IN) region of the HIV-1 POL gene.

According to one embodiment of the present disclosure, the forward primer is any one of SEQ ID NOs: 1 to 4.

A HIV-1 drug resistance analysis assay kit to achieve the second aspect of the present disclosure comprises: the primer set for RT-PCR; and a primer set for sequencing for analyzing the nucleotide sequence of the HIV-1 POL gene.

According to one embodiment of the present disclosure, the primer set for sequencing is a forward primer set selected from the group consisting of SEQ ID NOs: 8 to 19 and combinations thereof and a reverse primer set selected from the group consisting of SEQ ID NOs: 20 to 29 and combinations of thereof.

According to one embodiment of the present disclosure, the forward primer set comprises: any one of SEQ ID NOs: 8 to 10; any one of SEQ ID NOs: 11 to 14; any one of SEQ ID NOs: 15 and 16; SEQ ID NO: 17; and any one of SEQ ID NOs: 18 and 19.

According to one embodiment of the present disclosure, the reverse primer set comprises: SEQ ID NO. 20; any one of SEQ ID NOs: 21 and 22; SEQ ID NO: 23; any one of SEQ ID NOS: 14 and 25; and any one of SEQ ID NOs: 26 to 29.

According to one embodiment of the present disclosure, the forward primer set comprises SEQ ID NOs: 8, 11, 15, 17, and 18, and the reverse primer set comprises SEQ ID NOs: 20, 21, 23, 24, and 26.

A method for analyzing a sample to determine resistance against an HIV-1 drug, to achieve the third aspect of the present disclosure, comprises: (a) obtaining at least one sample including HIV-1 RNA from a subject; (b) amplifying a target gene in the sample by using a primer set for RT-PCR, wherein the primer set for RT-PCR comprises a forward primer selected from the group consisting of SEQ ID NOs: 1 to 6 and a reverse primer represented by SEQ ID NO: 7; and (c) sequencing the amplified target gene using a sequencing primer set.

According to one embodiment of the present disclosure, the HIV-1 RNA included in the at least one sample comprises a POL gene.

According to one embodiment of the present disclosure, the method further comprises (d) determining the resistance of the sample against the HIV-1 drug by analyzing the nucleotide sequence obtained through step (c).

According to one embodiment of the present disclosure, step (d) comprises: a step of arranging the sequenced nucleotide sequence; and inputting the aligned nucleotide sequence into an HIV-1 drug resistance gene nucleotide sequence database for analysis of the nucleotide sequence.

### Advantageous Effects

The primer set, kit, and analysis method according to the present disclosure can achieve a similar level of reliability to that of a currently commercialized kit and can offer a significantly reduced price to a consumer compared to commercially available kits. For example, when using currently commercially available kits, a cost of about $140 or more is required per test, whereas when using the kit of the present disclosure, a cost of about $30 or less is expected to be required per test. That is, it is expected that the cost for test can be very significantly reduced.

In addition, when using the kit of the present disclosure, it is possible to identify mutations occurring in a specific region in the POL gene, which were not able to be identified with conventional kits.

In addition, since it is possible to amplify at least a portion of the PR region, at least a portion of the RT region, and at least a portion of the IN region of the POL gene in one step RT-PCR process using one primer set, the kit of the present disclosure is competitive compared to conventional kits in terms of time, cost, and effort for analysis.

### BRIEF DESCRIPTION OF DRAWINGS:

FIG. 1 is a schematic view illustrating a partial structure including a POL gene of HIV-1;

FIG. 2 illustrates a sequencing strategy for sequencing a target gene amplified according to the present disclosure;

FIG. 3 illustrates the electrophoresis results of PCR products amplified according to an embodiment of the present disclosure;

FIGS. 4a to 4c illustrate sequencing results obtained through the sequencing according to one embodiment of the present disclosure;

FIGS. 5a to 5c illustrate sequencing results obtained through the sequencing according to one embodiment of the present disclosure; and

FIGS. 6a to 6c illustrate sequencing results obtained through the sequencing according to one embodiment of the present disclosure.

The above and other objectives, features, and advantages of the present disclosure will be more clearly understood from the following detailed description taken in conjunction with the accompanying drawings, but the present disclosure is not limited thereto. In describing the present disclosure, when the detailed description of the relevant known technology is determined to unnecessarily obscure the gist of the present disclosure, the detailed description may be omitted.

Unless defined otherwise in this disclosure, all technical and scientific terms used in this disclosure have the same meaning as commonly understood by the ordinarily skill in the art.

As used herein, the term “target DNA”, “target RNA”, “target gene”, or “target region” refers to a nucleic acid that is targeted by DNA amplification. The target DNA or target RNA serves as a template for amplification in a PCR reaction or an RT-PCR reaction.

As used herein, the term “polynucleotide” refers to double-stranded DNA or cDNA, or single-stranded DNA or RNA, and, unless otherwise specified, the polynucleotide includes nucleotide analogs.

As used herein, the term “primer” refers to a single-stranded oligonucleotide capable of serving as an initiation point for template-directed DNA synthesis under suitable conditions in a suitable buffer at a suitable temperature.

As used herein, the term “oligonucleotide” may, in some cases, be used interchangeably with “primer” or “polynucleotide”. The terms “forward primer” and “reverse primer” refer to primers that bind to the 3′ and 5′ ends of a specific region of a template to be amplified by polymerase chain reaction, respectively.

The sequence of the primer does not need to have a sequence that is completely complementary to a partial sequence of the template, but it is sufficient if it is substantially complementary to the extent that it can hybridize with the template and perform the intrinsic action of the primer. Accordingly, the primer set according to one embodiment of the present disclosure may be a sequence that is perfectly complementary to the nucleotide sequence of the template. Alternatively, the primer set according to another embodiment may not be a sequence that is perfectly complementary to the nucleotide sequence of the template or may be a sequence that is substantially complementary within a range that does not interfere with amplification of the target gene of interest.

As used herein, the term “substantially complementary” means that two nucleic acid strands that are sufficiently complementary in sequence are annealed to form a stable duplex. The complementarity need not be perfect. For example, there may be several mismatches of base pairs between two nucleic acids. However, when the number of mismatches is so high that hybridization does not occur even under minimal stringent hybridization conditions, the sequences are not substantially complementary. When two sequences are interpreted as “substantially complementary to each other” in the present disclosure, the case means that the sequences are sufficiently complementary to hybridize to each other under selected reaction conditions, such as stringent hybridization conditions. The relationship between the sufficient complementarity of nucleic acids to achieve specificity and the stringency of hybridization is well known in the art. Two substantially complementary strands, for example, are perfectly complementary or may contain one or more mismatches that sufficiently allow differences between paired and unpaired sequences. Thus, a “substantially complementary” sequence may refer to a sequence having base pair complementarity to a certain sequence by at least 100%, 95%, 90%, 80%, 75%, 70%, 60%, 50%, or any intermediate percentage between any two numbers thereof in the double stranded region.

Oligonucleotides used in the present disclosure may be synthesized and prepared by an appropriate method (such as chemical synthesis) among methods known in the art. In addition, oligonucleotides may be commercially available.

### Primer Set

The present disclosure provides primer sets for HIV-1 drug resistance assays. The primer set is an RT-PCR primer set for amplifying a target gene having drug resistance or is a sequencing primer set for sequencing the amplified target gene.

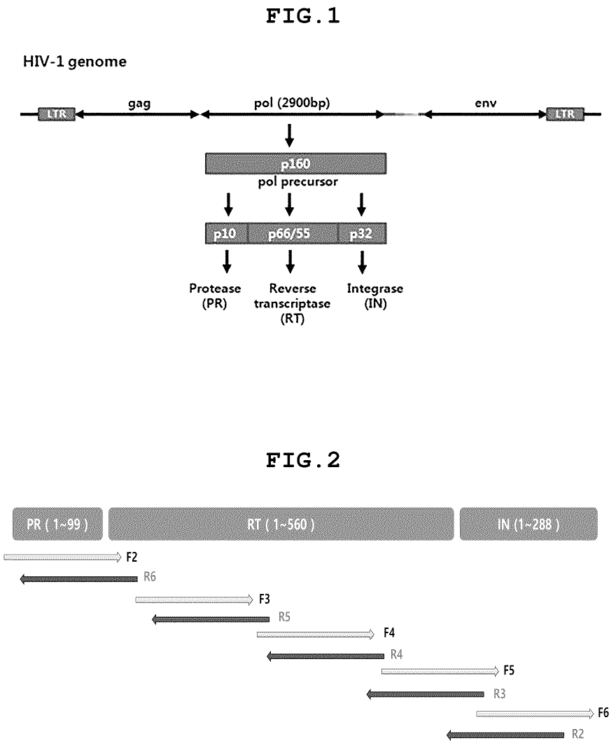

The RT-PCR primer set of the present disclosure can amplify a target gene having HIV-1 drug resistance. In the present disclosure, the target gene is a POL gene of HIV-1. As shown in FIG. 1, the POL gene is composed of a Protease (PR) region, a Reverse Transcriptase (RT) region, and an Integrase (IN) region.

In the present disclosure, the RT-PCR primer set simultaneously targets at least a portion of the Protease (PR) region, at least a portion of the Reverse Transcriptase (RT) region, and at least a portion of the Integrase (IN) region of the HIV-1 POL gene. Preferably, the RT-PCR primer set simultaneously may target the entire PR region, the entire RT region, and a portion of the IN region of the HIV-1 POL gene. According to one embodiment of the present disclosure, the primer set may be used to analyze codons 1 to 99 of the PR, codons 1 to 560 of the RT, and codons 1 to 285 of the IN as target regions. In the case of the currently commercialized primer set for diagnosing HIV-1 drug resistance of the RT region, there is a limitation in diagnosing only codons between 1 and 348. However, in the case of the primer set of the present disclosure, the primer set has an advantage in that it is possible to analyze the RT region exceeding number 348.

In addition, in the conventional art, there are only primer sets that target the PR, RT, and IN regions, respectively or are primer sets that simultaneously targets the PR and RT regions. However, there have been still no RT-PCR primers that target all of the PR, RT, and IN regions at the same time and which have good performance. However, the primer set of the present disclosure has achieved the functions. Accordingly, the primer set of the present disclosure does not require the use of multiple primer sets in diagnosing HIV-1 drug resistance and has advantages over existing primer sets in terms of time and cost for diagnosis.

The primer set of the present disclosure includes a forward primer and a reverse primer, and the forward primer may be selected from the group consisting of SEQ ID NOs: 1 to 6. In addition, the reverse primer may be shown as SEQ ID NO: 7.

TABLE 1

| SEQ ID NO | Primer | Sequence | mer |
|---|---|---|---|
| 1 | F1-1 | 5′-CAT AAG GCA AGA GTT TTG GCT GAA G-3′ | 25 |
| 2 | F1-2 | 5′-CCA TAA GGC AAG AGT TTT GGC TGA A-3′ | 25 |
| 3 | F1-3 | 5′-ATA AGG CAA GAG TTT TGG CTG AAG C-3′ | 25 |
| 4 | F1-4 | 5′-CAT AAR GCA AGA GTT TTG GCK GAA G-3′ | 25 |
| 5 | F1-5 | 5′-GCA GAG AGG CAA TTT TAG GAA CCA AAG-3′ | 27 |
| 6 | F1-6 | 5′-GCA ATG AGC CAA GTA ACA AAT TCA G-3′ | 25 |
| 7 | R1-1 | 5′-CCT GTC TAC TTG CCA CAC AAT CAT C-3′ | 25 |

The RT-PCR primer set may preferably be composed of sets shown below.

TABLE 2

| Set | Construction |
|---|---|
| set (1) | F1-1/R1-1 |
| set (2) | F1-2/R1-1 |
| set (3) | F1-3/R1-1 |
| set (4) | F1-4/R1-1 |

It is possible to amplify a target gene through RT-PCR using the primer sets described in Table 1 above. In addition, the nucleotide sequence of the amplified target gene may be analyzed using the DNA sequencing primer set of the present disclosure.

A sequencing primer set according to one embodiment of the present disclosure enables nucleotide sequence analysis of a target gene amplified by the RT-PCR primer set of the present disclosure. The sequencing primer set may include a forward primer set including a plurality of forward primers and a reverse primer set including a plurality of reverse primers. According to one embodiment of the present disclosure, the sequencing primer set may include a forward primer set including five forward primers and a reverse primer set including five reverse primers. Here, the target region of each of the five forward primers and the five reverse primers may partially overlap. This is to prevent omission of the sequencing.

FIG. 2 illustrates a sequencing strategy for sequencing a target gene amplified according to the present disclosure. Referring to FIG. 2, a plurality of forward primers (F2 to F6) and a plurality of reverse primers (R2 to R6) are used for sequencing without omission for all the regions including the PR region, RT region, and IN region, in which each arrow corresponds to a nucleotide sequence range that can be amplified by the corresponding primer. For example, it is shown that a region amplified by F5 may be at least partially overlap with a region amplified by R3, F6, or R2.

Through this, it is possible to prevent missing of the sequencing of the target region from occurring during sequencing. Specifically, by adopting two pairs of forward and reverse primers, such as F2 and R6, F3 and R5, F4 and R4, F5 and R3, and F6 and R2 whose target regions overlap, even though either the forward or reverse primer does not react during actual analysis, good sequencing results can be obtained because the remaining primer reacts. That is, this method has an advantage in terms of complementarity. According to one embodiment of the present disclosure, the amplification by the ten primers may be independently performed.

Table 3 below shows specific examples of primers included in the sequencing primer set of the present disclosure.

TABLE 3

| SEQ ID NO | Primer | Sequence | mer |
|---|---|---|---|
| 8 | F2-1 | 5′-TGT TGG AAA TGT GGA AAG GAA GGA C-3′ | 25 |
| 9 | F2-2 | 5′-ACA CCA AAT GAA AGA TTG TAC TGA G-3′ | 25 |
| 10 | F2-3 | 5′-TGA AAG ATT GTA CTG AGA GAC AGG C-3′ | 25 |
| 11 | F3-1 | 5′-AGA AAT TTG TAC AGA RAT GG-3′ | 20 |
| 12 | F3-2 | 5′-CAA TAC TCC AGT ATT TGC C-3′ | 19 |
| 13 | F3-3 | 5′-TCA AGA CTT CTG GGA AGT TC-3′ | 20 |
| 14 | F3-4 | 5′-GGG AAG TTC AAT TAG GAA TAC-3′ | 21 |
| 15 | F4-1 | 5′-ATA AAT GGA CAG TAC AGC C-3′ | 19 |
| 16 | F4-2 | 5′-AGC TGG ACT GTC AAT GAC-3′ | 18 |
| 17 | F5-1 | 5′-CAG GAT ATG TTA CTR AYA GAG GAA G-3′ | 25 |
| 18 | F6-1 | 5′-TGC ATG GAC AAG TAG ACT G-3′ | 19 |
| 19 | F6-2 | 5′-GCC AGT GGA TAT ATA GAA GC-3′ | 20 |
| 20 | K2-1 | 5′-CTT TAG TTT GTA TGT CTG TTG C-3′ | 22 |
| 21 | R3-1 | 5′-CTG CTG GAA TAA CTT CTG C-3′ | 19 |
| 22 | R3-2 | 5′-CCT TCT AAA TGT GTA CAA TCT AG-3′ | 23 |
| 23 | R4-1 | 5′-RAC AAA CTC CCA CTC AGG-3′ | 18 |
| 24 | R5-1 | 5′-AAT GGA GGT TCT TTC TGA TG-3′ | 20 |
| 25 | R5-2 | 5′-TTT GTC TGG TGT GGT AAR-3′ | 18 |
| 26 | R6-1 | 5′-GTG GTA TTC CTA ATT GAA CTT C-3 | 22 |
| 27 | R6-2 | 5′-CCT AAT TGA ACT TCC CAG-3′ | 18 |
| 28 | R6-3 | 5′-TAT GCA TCA CCC ACA TCC AG-3′ | 20 |
| 29 | R6-4 | 5′-GGA TGT GGT ATT CCT AAT TG-3′ | 20 |

Referring to Table 3, the forward primer set included in the sequencing primer set of the present disclosure may be selected from the group consisting of SEQ ID NOs: 8 to 19 and combinations thereof. In addition, the reverse primer set included in the sequencing primer set of the present disclosure may be selected from the group consisting of SEQ ID NOs: 20 to 29 and combinations thereof.

According to one embodiment of the present disclosure, the forward primer set may include: any one of SEQ ID NOs: 8 to 10; any one of SEQ ID NOs: 11 to 14; any one of SEQ ID NOs: 15 and 16; SEQ ID NO: 17; and any one of SEQ ID NOs: 18 and 19. More preferably, in terms of sequencing reliability, the forward primer set may include SEQ ID NOs: 8, 11, 15, 17, and 18.

According to one embodiment of the present disclosure, the reverse primer set may include: SEQ ID NO20; any one of SEQ ID NOs: 21 and 22; SEQ ID NO: 23; any one of SEQ ID NOs: 24 and 25; and any one of SEQ ID NOs: 26 to 29. More preferably, in terms of sequencing reliability, the reverse primer set may include SEQ ID NOs: 20, 21, 23, 24, and 26.

### Kit

The present disclosure provides a HIV-1 drug resistance analysis kit. The kit may include an RT-PCR primer set and a sequencing primer set. The RT-PCR primer set may be the RT-PCR primer set of the present disclosure described above. The sequencing primer set for analyzing the nucleotide sequence of the HIV-1 POL gene may also be the sequencing primer set of the present disclosure described above.

According to one embodiment of the present disclosure, the kit may include: an RT mixture including a reverse transcriptase, dNTP, and a buffer solution; a PCR mixture including a DNA polymerase, dNTP, and a buffer solution; nuclease-free water, or any combination of these. The RT mixture may be used to reverse transcribe RNA into DNA prior to amplification of a target gene, and the PCR mixture may be used to amplify DNA generated through the reverse transcription.

According to another embodiment of the present disclosure, the kit may further include a fluorescent dye, ddNTPs, a sequencing buffer solution, a purification reagent, or a combination of these. The PCR product may be purified before and after attaching a fluorescent dye to the target gene using the purification reagent. In the present disclosure, the purification reagent is, for example, shrimp alkaline phosphatase (SAP); Exonuclease I (EXO I); EtOH; sodium acetate; EDTA; or the like. A PCR product labeled with a fluorescent dye after being purified with the purification reagent may be subjected to nucleotide sequence analysis using a sequencer in a subsequent step.

### Analysis Method

The present disclosure provides a novel method for assaying HIV-1 drug resistance in a sample obtained from an HIV-1 infected person. The above-described primer set and/or kit of the present disclosure may be used in the method.

The method includes obtaining from at least one sample containing HIV-1 RNA from a subject. The HIV-1 RNA may contain a POL gene. The sample may be obtained by a known method from a body fluid already collected from a subject. According to one embodiment of the present disclosure, the body fluid may be blood, and, among them, may be specifically plasma separated from blood.

In addition, the analysis method of the present disclosure includes amplifying a gene targeted for HIV-1 drug resistance diagnosis in the sample. According to one embodiment of the present disclosure, the amplifying step may be performed through RT-PCR, and thus the above-described RT-PCR primer set of the present disclosure may also be used in the amplifying step.

In the RT-PCR, a sample containing HIV-1 RNA is reverse transcribed into cDNA by a reverse transcriptase, in which the reverse transcriptase may be a known reverse transcriptase and is not particularly limited. In one embodiment according to the present disclosure, the reverse transcription step is performed at a temperature of about 40° C. to 70° C., preferably about 45° C. to 60° C. for about 5 to 20 minutes, preferably about 5 to 15 minutes, and most preferably for about 10 minutes.

According to one embodiment of the present disclosure, the method may include a reverse transcription inactivation step and an initial denaturation step before entering the amplification of the cDNA generated after the reverse transcription step. The reverse transcription inactivation step inactivates the reverse transcriptase used in the reverse transcription step to prevent unnecessary reactions from occurring in the subsequent step, and the initial denaturation step means denaturing the generated cDNA so that the cDNA can be amplified in a subsequent step. The reverse transcription inactivation step and the initial denaturation step may be performed simultaneously, for example, in the temperature range of about 95° C. to 100° C. for a period of time in the range of about 1 minute 30 seconds to 2 minutes 30 seconds.

The amplifying step includes: i) a denaturation step of separating a double-stranded DNA into single-stranded DNAs; ii) annealing to binding the single-stranded denatured DNAs with a primer; iii) extending the bound primer.

Unlike a conventional art in which one of the PR, RT, and IN regions is targeted at one time, the amplification target region of the present disclosure includes at least a portion of the PR region, at least a portion of the RT region, and at least a portion of the IN region. Thus, The amplification step of present disclosure may require different conditions than the conventional one.

According to one embodiment of the present disclosure, the denaturing step may be performed for 5 to 20 seconds at a temperature in the range of 90° C. to 100° C. Preferably, the denaturing step may be performed for 7 seconds to 15 seconds at a temperature in the range of 95° C. to 100° C., and more preferably, the denaturing step is performed for 8 seconds to 12 seconds at a temperature in the range of 97° C. to 99° C.

According to one embodiment of the present disclosure, the annealing step may be performed for 5 to 20 seconds at a temperature in the range of 50° C. to 80° C. Preferably, the annealing step may be performed for 7 seconds to 15 seconds at a temperature in the range of 55° C. to 75° C., and more preferably, the annealing step may be performed for 8 seconds to 12 seconds at a temperature in the range of 55° C. to 72° C.

According to one embodiment of the present disclosure, the extending step may be performed for 90 seconds or more at a temperature in the range of 60° C. to 80° C. Preferably, the extending step may be performed for 100 seconds to 140 seconds at a temperature in the range of 65° C. to 75° C., and more preferably, the extending step may be performed for 110 seconds to 130 seconds at a temperature in the range of 70° C. to 74° C. Most preferably, the extending step may be performed at a temperature of about 72° C. for about 120 seconds.

According to one embodiment of the present disclosure, the denaturing step, the annealing step, and the extending step may be sequentially performed. When one cycle consists of one denaturing step, one annealing step, and one extending step, the steps maybe performed by 30 to 50 cycles, preferably 32 to 45 cycles, and more preferably 35 to 45 cycles.

In the present disclosure, the target gene to be amplified through RT-PCR is a gene including PR, RT, and IN regions having a long sequence length of about 3 kb. Therefore, the temperature and time conditions of each step are especially important. Among the conditions, the conditions for the reverse transcription step and the annealing step are the most important. When the temperature in the steps is out of the temperature range, it may be difficult to amplify a target gene having a length of 3 kb.

In addition, the analysis method of the present disclosure includes sequencing the target gene amplified through the amplification step using the sequencing primer set. Accordingly, the sequencing primer set of the present disclosure, which is described above, may be used in the sequencing step. According to one embodiment of the present disclosure, the sequencing step may include: a cycle sequencing step of amplifying a sequencing target gene using a sequencing primer set and a fluorescent dye and labeling the sequencing target gene with the fluorescent dye; and a final sequencing step of analyzing the nucleotide sequence of the gene labeled with the fluorescent dye using a sequencer or the like.

The cycle sequencing step may also include i) a denaturing step; ii) an annealing step; and iii) an extending step.

According to one embodiment of the present disclosure, the denaturing step may be performed for 5 to 20 seconds within in a temperature range of 90° C. to 100° C. Preferably, the denaturing step may be performed for 7 seconds to 15 seconds at a temperature in the range of 95° C. to 100° C., and more preferably, the denaturing step is performed for 8 seconds to 12 seconds at a temperature in the range of 95° C. to 97° C.

According to one embodiment of the present disclosure, the annealing step may be performed for 1 to 15 seconds at a temperature in the range of 40° C. to 60° C. Preferably, the annealing step may be performed for 3 seconds to 10 seconds at a temperature in the range of 45° C. to 55° C., and more preferably, the annealing step may be performed for 4 seconds to 6 seconds at a temperature in the range of 47° C. to 52° C.

According to one embodiment of the present disclosure, the extending step may be performed for 3 to 10 minutes at a temperature in the range of 50° C. to 70° C. Preferably, the extending step may be performed for 3 to 6 minutes at a temperature in the range of 55° C. to 65° C., and more preferably, the extending step may be performed for a period of time in the range of 3 minutes to 4 minutes 30 seconds at a temperature in the range of 58° C. to 62° C.

According to one embodiment of the present disclosure, the denaturing step, the annealing step, and the extending step included in the cycle sequencing step may be sequentially performed. When one cycle consists of one denaturing step, one annealing step, and one extending step, the steps maybe performed by 25 to 40 cycles, preferably 25 to 35 cycles, and more preferably 25 to 30 cycles.

According to one embodiment of the present disclosure, the analysis method may further include purifying the amplification product of the target gene obtained in the amplifying step, in which the purifying is performed after the amplifying step and before the cycle sequencing step. In the purification step, the remaining RT-PCR primers may be removed with a purification reagent, in which SAP and/or EXO may be used as the purification reagent. In addition, if necessary, the purified amplification product may be diluted with sterile distilled water before undergoing the cycle sequencing step. Similarly, the analysis method may further include a step of purifying the gene labeled with the fluorescent dye using the purification reagent, in which the gene purification may be performed after the cycle sequencing step. Here, as the purification reagent, for example, EtOH and/or sodium acetate, EDTA, etc. may be used.

After the cycle sequencing step, the analysis method of the present disclosure may analyze a specific nucleotide sequence of the gene labeled with the fluorescent dye through a final sequencing step. The final sequencing step may be performed using a commercially available sequencer device.

On the other hand, the analysis method of the present disclosure may further include determining the resistance of the sample to the HIV-1 drug on the basis of the nucleotide sequence analyzed through the sequencing step, in which the determining may be performed after the sequencing step.

According to one embodiment of the present disclosure, when the sequencing primer set of the present disclosure is used, a forward primer set including five forward primers and a reverse primer set including five reverse primers may be used. Accordingly, the determining of the resistance of the sample to the HIV-1 drug using the nucleotide sequence analyzed through the sequencing step includes: arranging the nucleotide sequence sequenced with each of these primers into one nucleotide sequence; and inputting the arranged nucleotide sequence into an HIV-1 drug resistance gene nucleotide sequence database for analysis. The database is not particularly limited as long as it is possible to determine the presence or absence of resistance to the HIV-1 drug targeted by the present disclosure. For example, the database “https://hivdb.stanford.edu/”, etc. may be used.

Hereinafter, preferred examples are presented to help the understanding of the present disclosure, but the following examples are provided only for easier understanding of the present disclosure, and thus the present disclosure is not limited thereto.

### EXAMPLE

### 1. Amplification of HIV-1 Target Gene

HIV-1 RNA (Samples 1 to 14) isolated from a sample (plasma) collected from an HIV-1 infected person was prepared in advance and stored frozen in a cooling device. After complete thawing on ice or laptop cooler, an RT-PCR mixture containing the HIV-1 RNA was prepared. The composition of the RT-PCR mixture is shown in Table 4 below.

TABLE 4

| Element | Volume |
|---|---|
| Nuclease-free water | 12.5 μl |
| 2X Platinum ™ SuperFi ™ RT-PCR | 25 μl |
| Master Mix | |
| Forward primer for RT-PCR | 1 μl (0.2 μM) |
| (10 μM) | |
| Reverse primer for RT-PCR | 1 μl (0.2 μM) |
| (10 μM) | |
| SuperScript ™ IV RT Mix | 0.5 μl |
| template (RNA) | 10 μl |
| Total | 50 μl |

Types of the forward and reverse primers are shown in Table 5 below.

TABLE 5

| Example | Set | Construction |
|---|---|---|
| Example 1 | set (1) | F1-1/R1-1 |
| Example 2 | set (2) | F1-2/R1-1 |
| Example 3 | set (3) | F1-3/R1-1 |
| Example 4 | set (4) | F1-4/R1-1 |

RT-PCR was performed, according to the RT-PCR reaction conditions as shown in Table 6 below, on RT-PCR mixtures containing Samples 1 to 14, respectively, using each of the RT-PCR primer sets of Examples 1 to 4. A Veriti 96-well Thermal Cycler was used as a PCR instrument.

TABLE 6

| Step | Temperature(° C.) | Time | | Number of cycles |
|---|---|---|---|---|
| 1 | 55 | 10 | min | 1 |
| 2 | 98 | 2 | min | |
| 3 | 98 | 10 | sec | 40 |
| 4 | 60 | 10 | sec | |
| 5 | 72 | 2 | min | |
| 6 | 72 | 5 | min | 1 |

| | | | |
|---|---|---|---|
| 7 | 4 | ∞ | 1 |

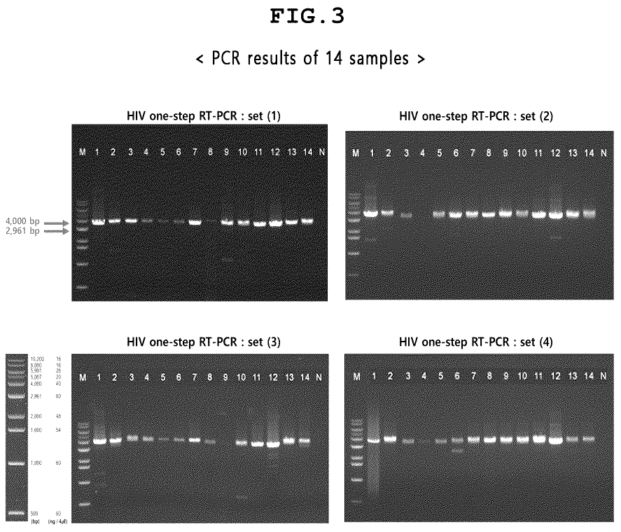

Next, each of the PCR products of Examples 1 to 4 was electrophoresed using a BIO-RAD's Gel Doc XR+ Gel Documentation System. The results are shown in FIG. 3. Referring to FIG. 3, as a result of the electrophoresis, it was confirmed that a PCR product of about 3200 bp was obtained through amplification using the primer sets of Examples 1 to 4 for all of Samples 1 to 14.

Accordingly, it can be seen that it is possible to amplify a target gene having a size of about 3 kb including all of the PR, RT, and IN regions of the POL gene of HIV-1 with the use of the primer set.

### 2. Purification of Amplification Product

With the amplification product obtained according to the method described in the section titled “1. Amplification of HIV-1 Target Gene”, a mixture having the composition shown in the following table was prepared.

TABLE 7

| Element | Volume |
|---|---|
| Amplification product | 10.0 μl |
| EXO (20 U/μl) | 0.5 μl |
| SAP(1 U/μl) | 1.0 μl |
| Nuclease-free water | 3.5 μl |
| Total | 15.0 μl |

The mixture was purified using a Thermal Cycler under the conditions shown in Table 8 below.

TABLE 8

| Step | Temperature (° C.) | Time |
|---|---|---|
| 1 | 37 | 45 min |
| 2 | 80 | 15 min |
| 3 | 4 | ∞ |

Next, the purified amplification product was diluted with distilled water so that the band intensity became 20 to 50 ng prior to the sequencing.

### 3. Sequence Analysis of Amplified HIV-1 Target Gene

1 μl of the diluted amplification product was mixed with 9 μl of each of the HIV-1 sequencing mixtures including forward primers F2-1, F3-1, F4-1, F5-1, and F6-1 and reverse primers R2-1, R3-1, R4-1, R5-1, and R6-1, respectively, and cycle sequencing was performed using a thermal cycler. The compositions of the HIV-1 sequencing mixtures are shown in Table 9 below.

TABLE 9

| Element | Volume |
|---|---|
| 5X Sequencing Buffer | 1.75 μl |
| BigDye ™ Terminator v1.1 or | 0.5 μl |
| v3.1 | |
| 2 um of one of primers | 1 μl |
| F2-1, F3-1, F4-1, F5-1, | |
| F6-1, R2-1, R3-1, R4-1, | |
| R5-1, R6-1 | |
| DW | 5.75 μl |
| Total | 9 μl |

The cycle sequencing was performed under the conditions of Table 9 below.

TABLE 10

| Step | Temperature(° C.) | Time | | Number of cycles |
|---|---|---|---|---|
| 1 | 96 | 1 | min | 1 |
| 2 | 96 | 10 | sec | 25 |
| 3 | 50 | 5 | sec | |
| 4 | 60 | 4 | min | |

| | | | |
|---|---|---|---|
| 5 | 4 | ∞ | 1 |

Next, 1 μl of 3M sodium acetate, 1 μl of 125 mM EDTA, and 25 μl of 100% ethanol were added to 10 μl of the obtained cycle sequencing PCR product. After centrifugation at 2000×g at 4° C. for 30 minutes, the supernatant was discarded, and 100 μl of 70% ethanol was added the precipitate. Next, centrifugation was performed again at 2000×g at 4° C. for 10 minutes. In order to completely remove the remaining ethanol, the plate or tube was inverted, and centrifugation was performed again at 150×g at 4° C. for 30 seconds. Next, the sample was dried at room temperature. 10 μl of Hi-Di formamide was added to the sample, mixed well, reacted at 95° C. for 2 minutes, and cooled on ice. The samples were placed in a sequencing analyzer and final sequencing was performed. As the sequencing analyzer, ABI 3130xL Genetic Analyzer or ABI 3500xL Dx Genetic Analyzer was used.

By inputting the nucleotide sequence obtained through the nucleotide sequence analysis into a database provided by “https://hivdb.stanford.edu/”, it is possible to identify the mutation site of the sample and to determine whether the sample is resistant to the HIV-1 drug.

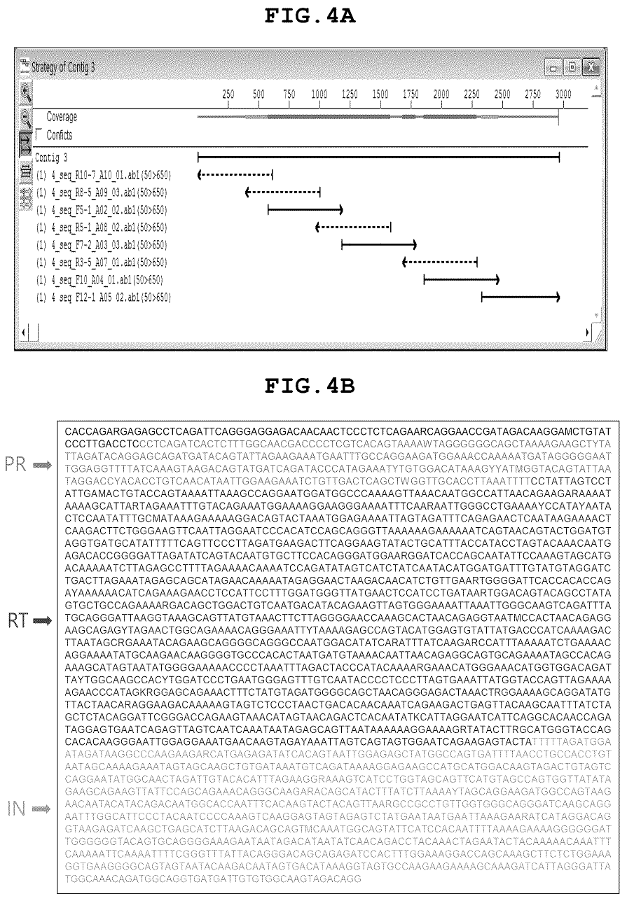

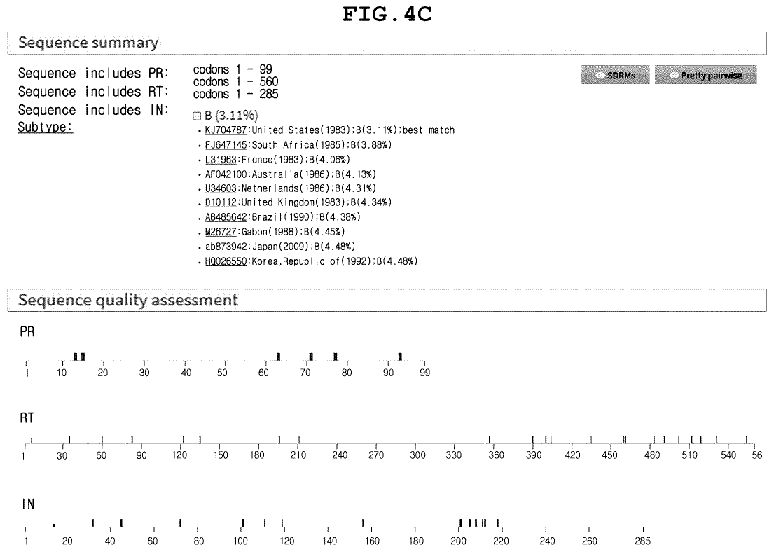

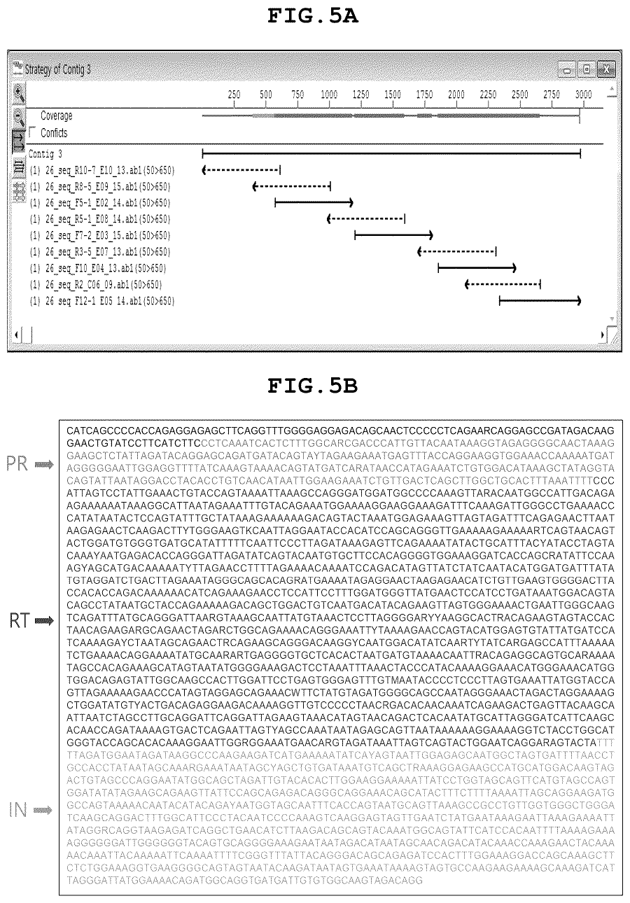

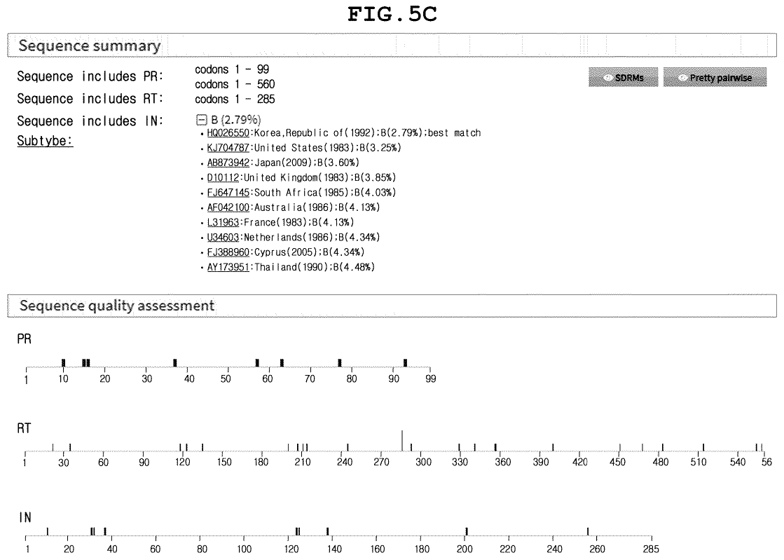

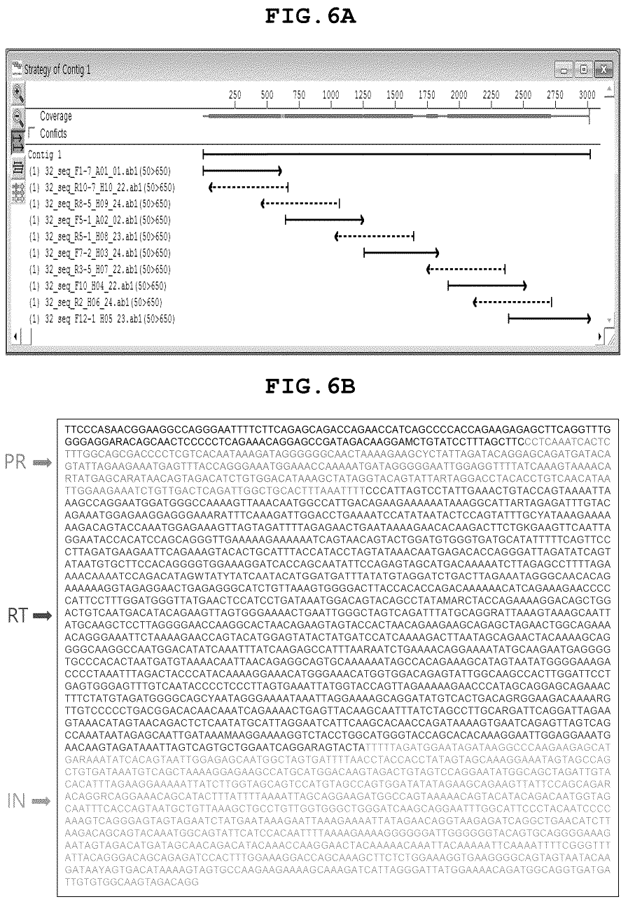

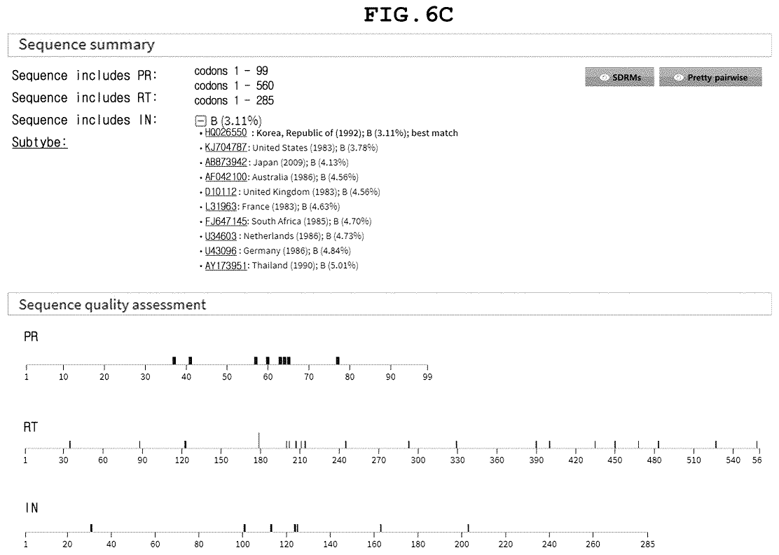

Illustratively, the results of sequencing and drug resistance analysis for the amplification products obtained by amplifying samples 1, 9, and 12 using the primer set (1) of Example 1 are shown in FIGS. 4 to 6 in order. Referring to FIGS. 4a to 6a , it is confirmed that sequencing was performed without missing any portion for a region of about 3200 bp. In addition, referring to FIGS. 4b to 6b, it is seen that all three targeted regions PR, RT, and IN were amplified with a single primer set and were smoothly sequenced with the sequencing primer set. In addition, as shown in FIGS. 4c to 6c, using the RT-PCR primer set and the sequencing primer set of the present disclosure instead of the conventionally commercialized diagnostic kit, it is confirmed that the diagnosis of HIV-1 drug resistance of the sample can be performed.

The results described above shows that the use of the primer set, kit, and analysis method of the present disclosure are expected to have significantly superior advantages over conventionally commercialized diagnostic kits in terms of time, cost, and performance, and particularly in terms of time and cost.

All simple modifications and alterations of the present disclosure fall within the scope of the present disclosure, and the specific protection scope of the present disclosure will be clearly defined by the appended claims.

1. An RT-PCR primer set for diagnosing HIV-1 drug resistance, the RT-PCR primer set comprising: a forward primer selected from the group consisting of SEQ ID NOs: 1 to 6; and a reverse primer represented by SEQ ID NO: 7.

2. The RT-PCR primer set of claim 1, wherein the primer set simultaneously targets the entire area of a protease (PR) region, the entire area of a reverse transcriptase (RT) region, and at least a portion of an integrase (IN) region of a HIV-1 POL gene.

3. The RT-PCR primer set of claim 1, wherein the forward primer is any one of SEQ ID NOs: 1 to 4.

4. A kit for diagnosing HIV-1 drug resistance, the kit comprising: the RT-PCR primer set of any one of claims 1 to 3; and a sequencing primer set for analyzing the nucleotide sequence of the HIV-1 POL gene.

5. The kit of claim 4, wherein the sequencing primer set comprises a forward primer set selected from the group consisting of SEQ ID NOs: 8 to 19 and combinations thereof and a reverse primer set selected from the group consisting of SEQ ID NOs: 20 to 29 and combinations of thereof.

6. The kit of claim 5, wherein the forward primer set comprises: any one of SEQ ID NOs: 8 to 10; any one of SEQ ID NOs: 11 to 14; any one of SEQ ID NOs: 15 and 16; SEQ ID NO: 17; and any one of SEQ ID NOs: 18 and 19.

7. The kit of claim 5, wherein the reverse primer set comprises: SEQ ID NO. 20; any one of SEQ ID NOs: 21 and 22; SEQ ID NO: 23; any one of SEQ ID NOs: 24 and 25; and any one of SEQ ID NOs: 26 to 29.

8. The kit of claim 5, wherein the forward primer set comprises SEQ ID NOs: 8, 11, 15, 17, and 18, and the reverse primer set comprises SEQ ID NOs: 20, 21, 23, 24, and 26.

9. A method for analyzing a sample to determine resistance against an HIV-1 drug, the method comprising: (a) obtaining at least one sample including HIV-1 RNA from a body fluid already collected from a subject; (b) amplifying a target gene in the sample by using an RT-PCR primer set, wherein the RT-PCR primer set comprises a forward primer selected from the group consisting of SEQ ID NOs: 1 to 6 and a reverse primer represented by SEQ ID NO: 7; and (c) sequencing the amplified target gene using a sequencing primer set.

10. The method of claim 9, wherein the HIV-1 RNA included in the at least one sample comprises a POL gene.

11. The method of claim 9, further comprising (d) determining whether the sample has resistance to an HIV-1 drug by analyzing the nucleotide sequence sequenced through step (c).

12. The method of claim 11, wherein step (d) comprises: arranging the sequenced nucleotide sequence; and inputting the arranged nucleotide sequence into an HIV-1 drug resistance gene nucleotide sequence database and analyzing the sequence.
